(19) United States
(12) Patent Application Publication
(10) Pub. No.: US 2022/0071531 A1
(43) Pub. Date: Mar. 10, 2022

(54) BLOOD COLLECTION SYRINGE

(71) Applicant: Conrad Flick, Bettendorf, IA (US)

(72) Inventor: Conrad Flick, Bettendorf, IA (US)

(21) Appl. No.: 17/017,606

(22) Filed: Sep. 10, 2020

(51) Int. Cl. A61B 5/153; A61B 5/15

(52) U.S. Cl. CPC A61B 5/1535; A61B 5/150389; A61B 5/150473; A61B 5/150816; A61B 5/150572

(57) ABSTRACT

A blood collecting syringe that advantageously indicates whether the needle has correctly entered the blood vessel, typically a vein. Correct placement is determined when the user observes flashback, or blood entering the syringe. Flashback can be undetected in certain situations due to lack of pressure in the patients vascular system, typically the venous system. This syringe includes an internal, luminal region having an adjustable volume. When desired, the initial luminal volume can be increased to create a partial vacuum. This partial vacuum results in an improved detection of flashback.

### FIELD OF THE INVENTION

The present invention relates to a blood collection needle assembly and, more particularly, to a shieldable and selectively aspirable blood collection needle assembly with a flashback chamber for providing venous entry confirmation.

### DESCRIPTION OF RELATED ART

Needle assemblies are used for the collection of fluid specimens such as blood, from patients for subsequent analysis. Some of these needle assemblies are intended for use with an evacuated, fluid collection tube. These needle assemblies have a needle hub with a central lumen for receiving a single cannula, or providing fluid communication between separated, axially mounted distal and proximal cannulas. The single cannula includes a sufficiently sharp distal end extending beyond the hub to pierce the skin of the patient for venous access or other source of targeted fluid; and a proximal end configured to pierce the rubber stopper of the evacuated tube placing the tube in fluid communication with the patient's vasculature. This proximal end is typically covered by a needle pierceable, resealable sleeve frictionally attached at its open base to the proximal end of the hub. As the evacuated tube is pressed onto the needle assembly, this sleeve is axially compressed by the rubber stopper of the evacuated tube and punctured by the proximal end of the needle cannula. The assembly with two separate, distal and proximal cannulas have similar characteristics to the single cannula at their respective ends, but with a separation at their common inward ends to permit viewing a fluid sample provided by the venous blood pressure upon the cannula entering the patient's vasculature.

Determining sufficient access to the proper source of the fluid in the patient may be problematic. A typical blood collection procedure requires the phlebotomist to locate the entry site and then enter the vein with the distal end of the needle cannula. Venous entry at this stage may not be confirmed. If the phlebotomist is reasonably certain that the targeted blood vessel providing sufficient supply has been entered, an evacuated collection tube is inserted onto the proximal tip of the needle cannula. A flow of blood will be established due to the pressure differential in the evacuated collection tube and the patient's blood pressure. This may be the first indication of access to the targeted vessel. If blood is not present in the collection tub, the procedure must be repeated. The initial flow of blood may be slow due to the length and cross-sectional dimensions of the needle cannula, relative pressure levels, especially in a vein, and actual blood flow characteristics. These factors may lead to the phlebotomist determining the target vessel was not sufficient nor accessed properly and withdraw the needle to repeat the procedure causing the patient further trauma. In view of the above uncertainties, many fluid collection needle assemblies are provided with a transparent flashback chamber within the hub in fluid communication with the needle cannula. This chamber receives a portion of the blood flow shortly after a vein has been accessed properly.

The used blood collection assembly must be safely discarded to prevent any possible disease transmission to the health care provider by an accidental needle stick. Accordingly, it is desirable to shield the used needle cannula immediately after withdrawing it from the patient. Needle shields are in many forms. For example, the most common is a simple closed sleeve which frictionally engages the distal hub end. This type creates the greatest risk of an accidental needle stick during shielding if the sleeve is misaligned with the needle cannula, even versions with enlarged collars are not preferred. There are versions with axial, proximal retraction to expose the needle, and then telescoping distally for shield coverage of the needle. Shields of this type are safe and effective in most situations but may interfere with others. Some rotational shields are hinged via a living hinge or snapped into an integral fitting on the proximal hub end. This shield can safely be rotated from an open position exposing the needle cannula to a closed position shielding the needle cannula. These shields are effective and widely used, however can be cumbersome when maneuvering the blood collection needle assembly and vacuum collection tubes simultaneously.

The difficulties and limitations suggested in the preceding and the desired features are not intended to be exhaustive but rather are among many which tend to reduce the effectiveness and user satisfaction with previously known needle assemblies. Other noteworthy problems and limitations may also exist; however, those presented above should be sufficient to demonstrate that blood draw assemblies appearing in the past will admit to worthwhile improvement.

### SUMMARY OF THE INVENTION

The subject invention is directed to a selectively aspirable, shieldable, fluid collection needle assembly. The needle assembly includes a transparent or translucent, cylindrical hub with a fluid communication lumen extending between its proximal and distal ends. There are three luminal regions. The distal luminal region has a central, stepped diameter lumen; the proximal portion having the cannula diameter is to orient the needle cannula and a distal recess to accept an adhesive for securing the distal end of the needle cannula within the hub. The central luminal region's diameter being slightly larger than the needle cannula outside diameter to provide a flashback chamber in combination with an aperture in the cannula. The proximal luminal region consists of a larger cylinder in cooperation with an internal, disposable seal to form the active aspirating chamber. The luminal chambers in the central and proximal regions are in open, fluid communication. Two outward radial arms are integrally molded onto the distal body and extend proximally. During a blood draw procedure involving aspiration, the arms are pressed inward causing straightening and proximal lengthening; forcing the proximal, cylindrical seal rearward increasing the aspirating chamber's volume and thus creating a partial vacuum. The distal shield does not interfere with the usage of the device nor block the visual observation of the flashback chamber.

The needle assembly includes a double ended cannula with a midsection aperture partially through its diameter. The distal end of the needle cannula projects distally beyond the hub a sufficient distance for venous access with a tip sufficiently sharp to pierce the skin. The proximal end of the needle cannula is sleeved and projects proximally beyond the hub a sufficient distance to be in fluid communication with the vacuum collection tube once its sheath and collection tube's stopper are pierced. Two individual cannulas, distal and proximal, with the same tip characteristics may replace the double ended cannula. Their interior end separation serves as the aperture in the double ended cannula.

The needle assembly further includes a sealing member with a pierceable, resiliently resealable, proximally closed sleeve enclosing the proximal end of the needle cannula; and extending distally to become a cylindrical seal in a sealing engagement with the interior of the large proximal aspirating chamber. The midsection of the sleeve will have a wall thickness permitting axial collapse and a proximal section with a thickness to maintain its columnar profile.

The needle assembly of the subject invention further includes a partial, vacuum collection tube guide integrally molded onto the proximal hub end. This serves as a convenient guide for the insertion of the vacuum tube onto the proximal needle end, and also a protective shield for the proximal needle tip.

The needle assembly further includes a protective cannula sheath; which when fully rotated, not only reveals the cannula, but also completes the evacuated collection tube guide.

### DESCRIPTION OF THE DRAWINGS

FIG. 1 is an axonometric view of a needle assembly in accordance with preferred embodiment of the invention;

FIG. 2 is a rear, axonometric view of a needle assembly of FIG. 1 in a readied posture;

FIG. 3 is an exploded, axonometric view of a needle assembly of FIG. 1;

FIG. 4 is a longitudinal cross-sectional, side view of the needle assembly of FIG. 1;

FIG. 5 is a longitudinal cross-sectional, side view of the cylindrical body 11;

FIG. 6 is a longitudinal cross-sectional, side view of the sleeve 31;

FIG. 7 is a side view of sheath 60;

FIG. 8 is a transverse, cross-sectional view of the vertical securing tab 72;

FIG. 9 is an exploded axonometric view of a needle assembly in accordance with an alternative embodiment of the invention.

FIG. 10 is an axonometric view of a needle assembly 110 in a readied posture;

FIG. 11 is an exploded, axonometric view of a needle assembly of FIG. 9;

FIG. 12 is a longitudinal cross-sectional, side view of the needle assembly of FIG. 9.

### DETAILED DESCRIPTION

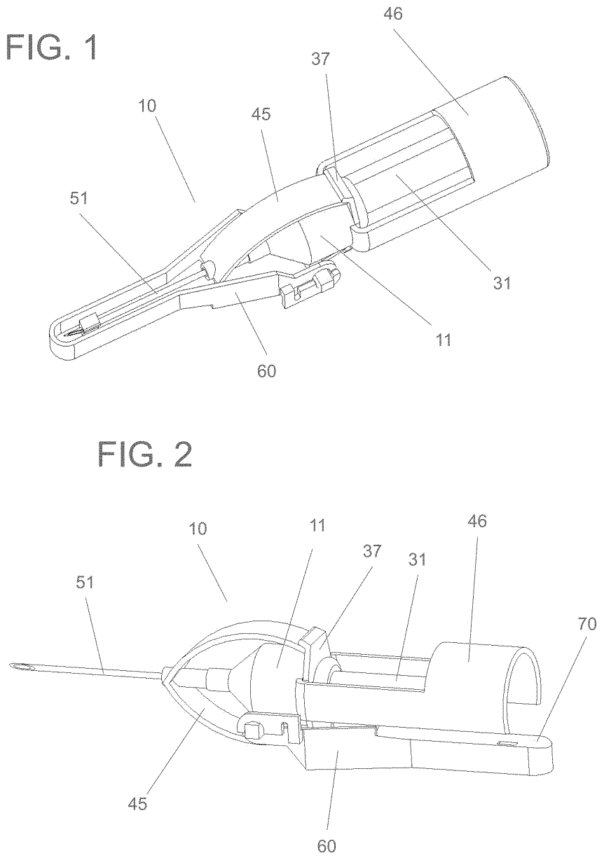

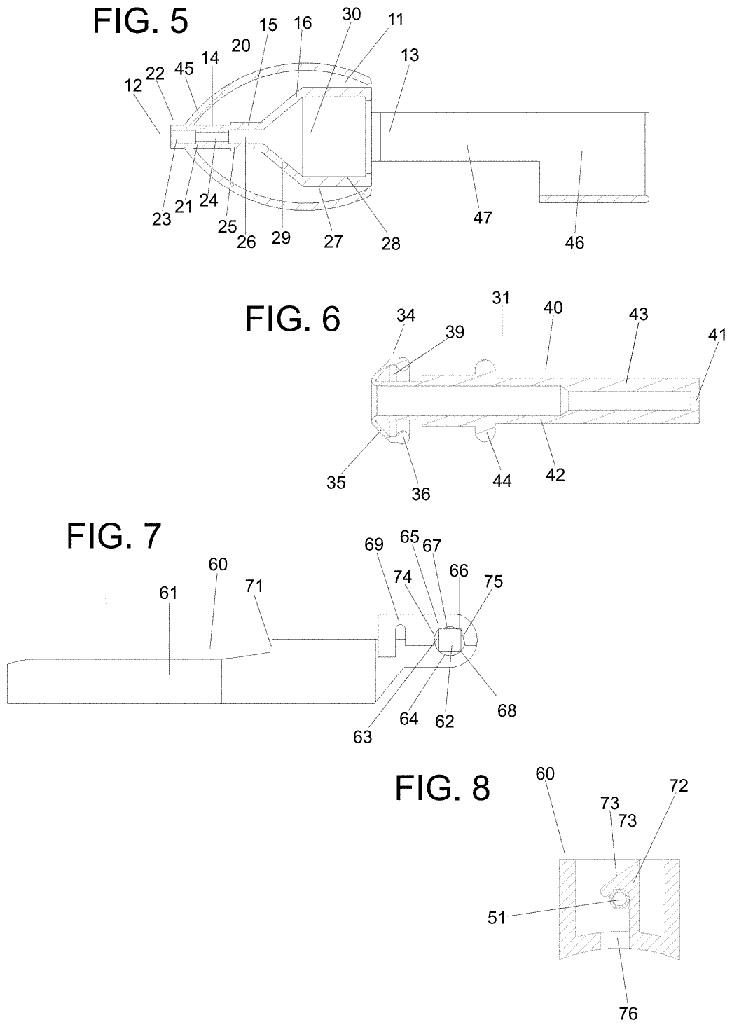

Referring now particularly to the drawings, wherein like reference characters refer to like parts, and initially to FIG. 1 and FIG. 2, there will be seen axonometric views of a needle assembly 10 in accordance with the preferred embodiment of the invention. The needle assembly 10 in FIG. 1 and FIG. 5 comprises a transparent or translucent, cylindrical hub 11 with a stepped, fluid communication lumen 20 extending between its distal and proximal ends, 12 and 13 respectively. There are three luminal regions. The distal luminal region 14 has a central, stepped diameter lumen 21; the distal portion 22 has a recess 23 to accept an adhesive for securing the distal end of the needle cannula 52 within the hub 11, and the proximal portion 24 orients the needle cannula 51 within its similar diameter. The central luminal region 15's diameter is slightly larger than the needle cannula 51's outside diameter to provide a flashback chamber 26 in cooperation with an aperture 56 in the cannula 51 for examination of patient's flashback or aspirated specimen. The proximal luminal region 16 consists of an enlarged cylinder 27 with a radially smooth, inner surface 28, and distally capped with a slight conical distal end wall 29. This conical profile permits the overall exposure and inward deformation of the distally convex, thin end wall 35 of the elastomeric chamber seal 34 with patient's blood pressure providing passive aspiration. This luminal enlargement 27 in cooperation with an inner, disposable seal 32 forms the active aspiration chamber 30. The luminal chambers in the central and proximal regions are in open, fluid communication. Two outward radial arms 45 are integrally molded onto the distal body end 12 and extend proximally. These arms 45 when inwardly compressed initiate active aspiration in cooperation with aspirating component 48. The pressure to compress the aspirating arms 45 is considerably greater than the grip pressure needed to accomplish venous entry with the IV cannula 52 permitting only selective aspiration. When relaxed after aspiration, the arms 45 return to their original posture leaving thimble 37 retaining the aspirating chamber seal 34 proximally displaced to prevent pressurizing the flashback chamber 26 and reintroducing aspirated fluid into patient. The aspirating component 31 can also be entirely manipulated manually.

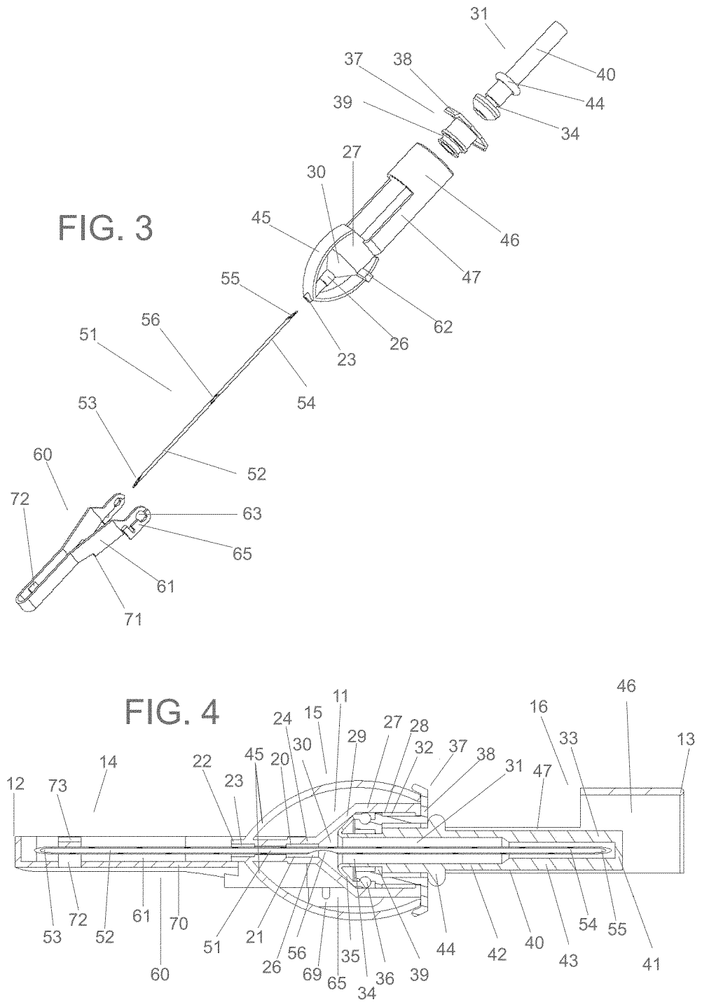

Referring to FIGS. 3,4, and 6, the aspirating component 48 includes a sealing member 31 consisting of two regions, distal and proximal, 32 and 33, respectively. The component's distal region 32 comprises an elastomeric, fluid tight, chamber seal 34 frictionally affixed to a support thimble 37 with complimentary, inner grooves 39. The patient distal region comprises a deformable, resiliently resealable sleeve 40 covering the patent distal cannula, or the non-patient (NP) cannula 54. The chamber seal 34's circumferential sealing ring 36 is in a fluid tight engagement with the inner surface 28 of the body's aspirating chamber 30. The convex distal wall 35 of the chamber seal 34 is inwardly deformable to allow passive aspiration with simply the patient's blood pressure. If selective, active aspiration is needed, the aspirating arms 45 are proximally extended with applied inward pressure causing the support thimble 37 to proximally displace chamber seal 34 via proximal pressure by thimble stop 38 on sleeve stop 44 increasing the aspirating chamber 30's volume. The presence of blood in the flashback or aspirating chambers 26 and 30, respectively, verifies proper IV cannula 52 placement in the patient's vasculature and the evacuated collection tube 82 can be installed. The luminal chambers 26 and 30 maintain their post-aspiration volume after disengaging from the retracting aspirating arms 45. When active aspiration takes place, the sealing component 31 moves proximally creating proximal separation between the NP cannula 54 and the proximal sleeve 40's pierceable end 41. The midsection of sleeve 40 will have a wall thickness 42 permitting axial collapse and a proximal wall thickness 43 sufficient to maintain its columnar profile ensuring cannular 51 penetration only of its pierceable, self-sealing end wall 41. The sleeve 40 contains the fluid accessed by the needle assembly 10 until the collection tube 82 is slid through guide 46 onto the NP cannula 54, axially collapsing and piercing the sleeve 39, and fully penetrating the stopper 83 to place the evacuated collection tube 82 in fluid communication with the needle cannula 51. The pressure differential between the collection tube 82 and the patient's vasculature establishes the fluid flow. If the procedure needs to be repeated, the phlebotomists simply removes needle assembly 10 from the patient; presses the thimble 37 to its initial position safely disposing aspirated material; and attempts to reestablish patient's venous access.

A partial, integral evacuated collection tube guide 46 is proximally poised with two opposing support arms 47 onto the proximal end of the aspirating chamber 30 in axial alignment with NP cannula 54.

The needle assembly 10 further includes a double ended needle 51 having a distal patient intravenous (IV) cannula 52; a proximal NP cannula 54; and an approximately central, aperture 56. The IV cannula projects distally beyond the body 11 a sufficient distance with a tip 53 beveled sufficiently sharp for patient venous access. The NP cannula tip 55 projects proximally beyond the body 11 a sufficient distance to be in fluid communication with the vacuum collection tube 82 once the self-sealing proximal sleeve 40 and the rubber stopper 83 are pierced. The aperture 56 permits the blood to enter the flashback chamber 26 to verify proper IV cannula 52 placement in the patient's vasculature.

Referring to FIG. 7, the needle assembly 10 further includes selectively rotatable, safety sheath 60. Its peripheral wall 61 outlines the IV section of the needle assembly 10 profile. It rotates about the square, sheath posts 62 integrally molded onto the body 11. The receiver slot 63 in the sidewall 61 has a curved bottom profile 64 for axial containment upon sheath 60 rotation. To establish sheath 60 position, the integrally molded, biased, containment levers 65 axially maintain and restrict sheath 60 rotation with partial square detents 66 in its upper curved profile 67 corresponding to the post corners 68. As rotation is initiated, these levers 65 flex upward freeing the partial detents 66 from the posts corners 68 and allowing sheath 60 rotation. The curved bottom profile 64 consists of two varying arcs 74 and 75 flanking detent 66 which tactily traces the post corner 62 as the levers 65′ trajectories expand upon the sheath 60's rotation for rotation containment. Levers 65 rotate via integrally molded living hinges 69 mutually attached to both the sheath sidewall 61 and levers 64. These hinges 69 return the levers 65 to their restricting engagement and rest position. The sheath 60 has an infill wall 70 at its distal end 12 complimenting the curvature of the partial, integral evacuated collection tube guide 46. The sampling positioned sheath 60 presents two collection tube 82 stops 71. Viewing FIG. 8, a deflectable, vertical tab 72 extends upward from the infill wall 70 to circumferentially engage IV cannula 52 to prevent inadvertent access. This catch 72 has a downward sloping top surface 73 to promote deflection by IV cannula 52 for safe stowing after use. To begin the draw procedure, simply move sheath 60 sideways slightly to access cannula 52 by rotating sheath 60.

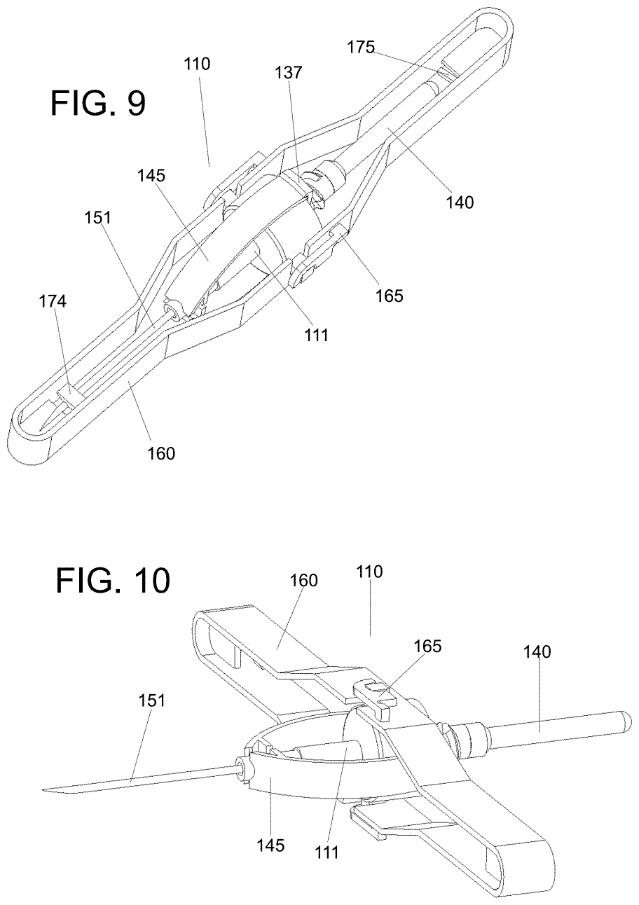

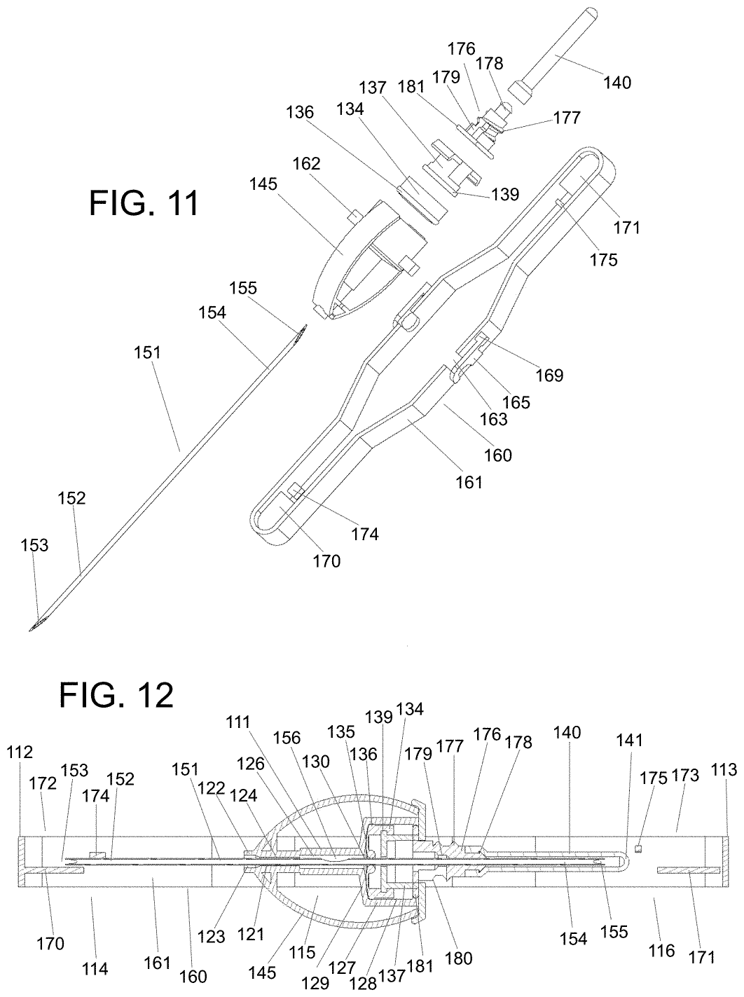

In FIGS. 9 and 10, there will be seen axonometric views of a needle assembly 110 in accordance with an alternative embodiment of the invention. The needle assembly 110 in FIGS. 11 and 12 comprises a transparent or translucent, cylindrical hub 111 with a stepped, fluid communication lumen 120 extending between its distal and proximal ends, 112 and 113 respectively. There are three luminal regions. The distal luminal region 114 has a central, stepped diameter lumen 121; the distal portion 122 has a recess 123 to accept an adhesive for securing the distal end of the needle cannula 152 within the hub 111, and the proximal portion 124 orients the needle cannula 151 within its similar diameter. The central luminal region 115's diameter 125 is slightly larger than the needle cannula's outside diameter to provide a flashback chamber 126 in cooperation with an aperture 156 in the cannula 151 for examination of patient's flashback or aspirated specimen. The presence of blood in the flashback 126 or aspirating 130 chambers verify proper IV cannula 152 placement in the patient's vasculature. The proximal luminal region 116 consists of an enlarged cylinder 127 with a radially smooth, inner surface 128, and distally capped with a slight conical distal end wall 129. This conical profile permits the overall exposure and inwardly deformation of the distally convex, thin end wall 135 of the elastomeric seal 134 with patient's blood pressure producing passive aspiration. This luminal enlargement 127 in cooperation with an inner, disposable seal 134 forms the active aspiration chamber 130. The luminal chambers in the central and proximal regions are in open, fluid communication. Two outward radial arms 144 are integrally molded onto the distal body 112 and extend proximally. During a blood draw procedure involving aspiration, the arms 144 are pressed inward causing straightening and proximal lengthening; forcing the retaining thimble 137 and cylindrical chamber seal 134 rearward, increasing the aspirating chamber's 130 volume. The pressure to compress the aspirating arms 144 is considerably greater than the grip pressure needed to accomplish venous entry with the IV cannula 152 permitting only selective aspiration. When relaxed after aspiration, the arms 144 return to their original posture leaving thimble 137 retaining the aspirating chamber seal 134 proximally displaced to prevent pressurizing the flashback chamber 126 and reintroducing aspirated fluid into patient. Integrally molded onto the exterior sidewall of the body 111 are two square posts 151 establishing the rotating axis of the sheath 160.

The needle assembly 110 further includes a double ended cannula 151 having a distal patient intravenous (IV) cannula 152; a proximal non-patient (NP) cannula 154; and an approximately central aperture 156. The IV cannula 152 projects distally beyond the hub 111 a sufficient distance with a tip 153 beveled sufficiently sharp for patient venous access. The NP cannula tip 155 projects proximally beyond the hub 111 a sufficient distance to be in fluid communication with the vacuum collection tube 182 once its self-sealing sleeve 139 and collection tube 182's stopper 183 are pierced. The aperture 156 permits the blood to enter the flashback chamber 126 to verify proper IV cannula 152 placement in the patient's vasculature. Two individual cannulas, distal and proximal, with the same tip characteristics may replace the double ended cannula. Their interior end separation serves as the aperture in the double ended cannula.

The needle assembly 110 further includes an aspirating component 131 consisting of an elastomeric, fluid tight seal 134 frictionally affixed to a support thimble 137 with complimentary, inner grooves 138. The seal's circumferential sealing ring 136 is in a fluid tight engagement with the inner surface 128 of the body's aspirating chamber 130. The convex, distal wall 135 of the seal 134 is inwardly deformable to allow passive aspiration with simply the patient's blood pressure. The displacement volume from deformation is greater than the negative space within the cannula 152. The luminal chambers 126 and 130 maintain their post-aspiration volume. If passive and active aspiration the procedure needs to be repeated, the phlebotomists simply removes needle assembly 110 from the patient; presses the thimble 137 to its initial position safely disposing aspirated material; and attempts to reestablish patient's venous access.

The needle assembly 110 further includes a proximal support fitting 176 affixed to the proximal end of the aspirating chamber 130 with a snap groove 181. Once in place, adhesive is placed in recess 179 to secure and seal the cannula 151. This fitting 176 supports a separate, vacuum collection tube guide (not shown) with an integral thread 177 and stop 180; and the pierceable, resiliently resealable sleeve 139 with a proximal, barbed groove 178. The stop 180 is also the possible engagement hub for a separate, protective, NP cannula 154 sheath.

The needle assembly 110 further includes a pierceable, resiliently resealable, proximally closed sleeve 139 covering the proximal end of the NP needle cannula 154 and engaging the proximal support fitting 176 in a fluid tight engagement. This sleeve contains the fluids accessed by the needle assembly 110 until the collection tube stopper 183 is slid onto the NP cannula 154 axially displacing columnar wall 142 by collapsing midsection wall 141, while piercing the sleeve end 140, and fully penetrating the stopper 183 to place the evacuated collection tube 182 in fluid communication with the needle cannula 151.

The needle assembly 110 further includes selectively rotatable, safety sheath 160. Its peripheral wall 161 outlines the total needle assembly 110 profile. It rotates about the square sheath posts 162 integrally molded onto the body 111. The receiver slot 163 in the sidewall 161 has a curved bottom profile 164 for axial containment upon sheath 160 rotation. To establish sheath 160 position, the integrally molded, biased, containment levers 165 axially maintain and restrict sheath 160 rotation with partial square detents 166 in its upper curved profile 167 corresponding to the post corners 168. As rotation is initiated, these levers 165 flex upward freeing the partial detents 166 from the posts 162 and allowing sheath 160 rotation. These levers 165 rotate on integrally molded, biased living hinges 169 mutually attached to both the sheath sidewall 161 and levers 165. These biased hinges 169 return the levers 165 to their restricting engagement and rest position. The sheath has two lateral, partial walls 170 and 171 infilling the shielding end 162 and the locking end 173. The shielding end 172 protects the IV cannula 152 in preparation for use. A horizontal tab 174 extends from one sheath sidewall past center to engage cannula 152 in resting position to prohibit its access. To begin the blood draw procedure, simply move sheath 160 sideways slightly to release IV cannula 152 by rotating sheath 160. The locking end 173 has a resiliently deflectable cannula lock 175 that is integrally angled from one sidewall towards the opposing sidewall and angling downwards. As the sheath 160 is rotated closed, the lock 175 yields to the contacting cannula 152 allowing passage, and then returns to its undeflected position to trap cannula 151. The sheath 160 does not interfere with the usage of the device 110 nor block the visual observation of the flashback 126 and aspirating 130 chambers.

The transition from passive aspiration to selective, active aspiration is performed smoothly and facilely with one hand without regripping. The various, above described features can be interchangeable within the various embodiments and utilize the various embodiments of the subject invention, a fluid drawing needle assembly with passive aspiration with the expansion of the inner chamber volume solely by the patient's blood pressure or selective, one handed, active aspiration.

1. A needle assembly comprising:
a transparent housing having a closed distal end and an open proximal end fluidly communicating via a stepped lumen, and
a distal intravenous cannula connected to a proximal, nonpatient cannula sharing at least one of an aperture and a separation and together projecting distally and proximally beyond said housing, and
a disposable liquid-tight seal occluding proximal lumen and enclosing the proximal, nonpatient cannula, and
a selective positional sheath visually non obstructing flashback or aspirating chambers.

2. The needle assembly as defined in claim 1 wherein said disposable, liquid-tight seal selectively travels proximally as in operable to increasing luminal volume.

3. The needle assembly as defined in claim 2 wherein proximal travel initiated by contact with outward arcuate arms lengthening with inward compression.

4. The needle assembly as defined in claim 1 wherein at least one of said seal and housing component being deformable supporting passive aspiration.

5. The needle assembly as defined in claim 1 wherein said biased, faceted, restricting levers integral to the sheath maintain tactile engagement with cornered, housing posts throughout rotation for movement isolation and positioning.

6. The needle assembly as defined in claim 1 wherein said proximally rotated sheath completes partial integrally molded vacuum tube guide.

7. The needle assembly as defined in claim 1 wherein at least one catch on sheath restrictively engages intravenous cannula preventing exposure.

8-9. (canceled)

10. A needle assembly comprising:
a transparent housing having a closed distal end and an open proximal end fluidly communicating via a stepped lumen, and
a distal intravenous cannula connected to a proximal, nonpatient cannula sharing at least one of an aperture and a separation and together projecting distally and proximally beyond said housing, and
a disposable liquid-tight seal occluding proximal lumen,
a collapsing, resiliently self-sealing sleeve enclosing the patient distal cannula, and
a selective positional sheath visually non-obstructing flashback or aspirating chambers.

11. The needle assembly as defined in claim 10 wherein said disposable, liquid-tight seal selectively travels proximally as in operable to increasing luminal volume.

12. The needle assembly as defined in claim 11 wherein proximal travel initiated by contact with outward arcuate arms lengthening with inward compression.

13. The needle assembly as defined in claim 10 wherein at least one of said seal and housing component being deformable supporting passive aspiration.

14. The needle assembly as defined in claim 10 wherein said sheath provides cannular shielding or locking postures with opposing rotations.

15. The needle assembly as defined in claim 10 wherein said biased, faceted, restricting levers integral to the sheath maintain tactile engagement with cornered, housing posts throughout rotation for movement isolation and positioning.

16. The needle assembly as defined in claim 10 wherein at least one catch on sheath restrictively engages intravenous cannula preventing exposure.
